(19) United States
(12) Patent Application Publication
(10) Pub. No.: US 2026/0293863 A1
(43) Pub. Date: Oct. 1, 2026

(54) VERIFICATION OF METHANE ABATEMENT IN ANIMALS

(71) Applicant: COMMONWEALTH SCIENTIFIC AND INDUSTRIAL RESEARCH ORGANISATION, Acton, Australian Capital Territory (AU)

(72) Inventors: Francesca MCINERNEY, Acton, Australian Capital Territory (AU); Nina WELTI, Acton, Australian Capital Territory (AU)

(73) Assignee: COMMONWEALTH SCIENTIFIC AND INDUSTRIAL RESEARCH ORGANISATION, Acton, Australian Capital Territory (AU)

(21) Appl. No.: 19/490,926

(22) PCT Filed: Jun. 6, 2024
(86) PCT No.: PCT/AU2024/050596

§ 371 Date: Dec. 6, 2025

(30) Foreign Application Priority Data
Jun. 7, 2023 (AU) 2023901808
Feb. 23, 2024 (AU) 2024900449

(51) Int. Cl. A01K 67/02; A23K 10/00; A23K 50/10; G01N 33/497; G01N 33/50; G16C 20/20

(52) U.S. Cl. CPC A01K 67/02; A23K 10/00; A23K 50/10; G01N 33/4975; G01N 33/50; G16C 20/20; A01K 2207/25; A01K 2227/10; G01N 2458/15

(57) ABSTRACT

This disclosure relates to a method for detecting methane reduction by using methane reduction means on ruminant livestock. The method comprises measuring a carbon isotope ratio in material from the ruminant livestock under use of the methane reduction means, to obtain a measured value and determining a reference value indicative of a reference carbon isotope ratio of material from the ruminant livestock without the use of the methane reduction means. The method further comprises determining a difference between (a) the reference value, and (b) the measured value; and based on the difference, detecting the methane reduction.

### CROSS-REFERENCE TO RELATED APPLICATIONS

The present application claims priority from Australian Provisional Patent Application No 2023901808 filed on 7 Jun. 2023, Australian Provisional Patent Application No 2024900998 filed on 10 Apr. 2024, the contents of which are incorporated herein by reference in their entirety.

### TECHNICAL FIELD

This disclosure relates to detecting methane emission reduction in ruminant livestock.

### BACKGROUND

Methane emissions from ruminant livestock is a large contributor to global warming. Therefore, there is a need to reduce methane emissions. While there are successful strategies to reduce methane emissions from ruminant livestock, it is difficult to detect whether those strategies have been applied so that reductions can be appropriately accounted for.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### SUMMARY

There is provided a method for detecting methane reduction by using methane reduction means on ruminant livestock. The method comprises:
measuring a carbon isotope ratio in material from the ruminant livestock under use of the methane reduction means, to obtain a measured value;
determining a reference value indicative of a reference carbon isotope ratio of material from the ruminant livestock without the use of the methane reduction means;
determining a difference between
   (a) the reference value, and
   (b) the measured value; and based on the difference, detecting the methane reduction.

In some embodiments, determining the reference value comprises measuring the reference carbon isotope ratio from the same individual from which the measured value is obtained on material grown at a time without the use of the methane reduction means.

In some embodiments, determining the reference value comprises measuring the reference carbon isotope ratio from a different individual that has been fed a comparable diet to the ruminant livestock without the use of the methane reduction means.

In some embodiments, the comparable diet is similar in relation to its carbon isotope ratio and/or abundance of C3 plant and C4 plant components.

In some embodiments, determining the reference value comprises:
measuring the reference carbon isotope ratio from an animal with a different diet; and
correcting the reference carbon isotope ratio for the different diet by calculating an isotopic composition of the diet and adjusting the reference carbon isotope ratio based on the isotopic composition.

In some embodiments, determining the reference value comprises measuring the reference carbon isotope ratio from a first tissue that is of a different tissue type than a second tissue from which the measured carbon isotope value is obtained.

In some embodiments, the method further comprises applying an offset to account for a constant difference in carbon isotope uptake in different tissues.

In some embodiments, determining the reference value comprises evaluating an uptake model, and an input to the uptake model comprises a diet of the ruminant livestock without the use of the methane reduction means.

In some embodiments, the method further comprises estimating the diet using geographic parameters.

In some embodiments, the material comprises one or more of: meat, wool, excrement, milk, bones, leather, hair, reproductive material or skin.

In some embodiments, the method further comprises evaluating a system model of the ruminant livestock.

In some embodiments, the system model is indicative of relative abundance of carbon isotopes entering the ruminant livestock and relative abundance of carbon isotopes exiting the ruminant livestock during the life of the ruminant livestock and relative abundance of carbon isotopes remaining in the material of the ruminant livestock.

In some embodiments, the system model is indicative of relative abundance of carbon isotopes entering the ruminant livestock and relative abundance of carbon isotopes exiting the ruminant livestock as gas during the life of the ruminant livestock and relative abundance of carbon isotopes ingested by the ruminant livestock.

In some embodiments, the method further comprises calculating a quantity of methane reduction based on the difference.

In some embodiments, calculating the quantity comprises evaluating a linear relationship representing a mixing model of carbon isotopes in a body of the ruminant livestock.

In some embodiments, the methane reduction means comprises a feed supplement.

In some embodiments, the method further comprises adjusting an amount of supplement fed to the ruminant livestock to achieve a desired detection or quantity of reduced methane.

There is provided a method for feeding a supplement to ruminant livestock at a dosage that causes a change in carbon isotope ratio in material from the animal indicative of a reduction in methane.

In some embodiments, the supplement comprises Asparagopsis.

A method for selective breeding comprises:
determining for multiple individuals an amount of methane emitted according to any one of the preceding claims;
using the values as phenotype for genetic and/or genomic evaluation; and
selecting individuals for further breeding based on the resulting breeding values.

There is provided an animal bred according to the above method.

There is further provided a kit comprising a feed supplement and instructions to feed the feed supplement to ruminant livestock for detection of methane emission.

There is further provided an animal product having a measured relative carbon isotope ratio difference indicative of a reduced methane emission as a result of feeding a supplement to the animal.

In some embodiments, the animal product is certified according to the method above.

There is further provided a computer system for detecting methane reduction by using methane reduction means on ruminant livestock, the computer system comprising one or more processors configured to:
receive a measured value indicative of a measurement of a carbon isotope ratio in material from the ruminant livestock under use of the methane reduction means;
determine a reference value indicative of a reference carbon isotope ratio of material from the ruminant livestock without the use of the methane reduction means;
determine a difference between
   (a) the reference value, and
   (b) the measured value; and based on the difference, detect the methane reduction.

There is further provided software that, when executed by one or more processors, causes the one or more processors to perform a method for detecting methane reduction by using methane reduction means on ruminant livestock, the method comprising:
receiving a measured value indicative of a measurement of a carbon isotope ratio in material from the ruminant livestock under use of the methane reduction means;
determining a reference value indicative of a reference carbon isotope ratio of material from the ruminant livestock without the use of the methane reduction means;
determining a difference between
   (a) the reference value, and
   (b) the measured value; and based on the difference, detecting the methane reduction.

### BRIEF DESCRIPTION OF DRAWINGS

FIG. 1 illustrates a method for detecting methane reduction using methane reduction means on ruminant livestock.

FIGS. 2a and 2b illustrate an animal update model without use of methane reduction means and with use of methane reduction means, respectively.

FIG. 3: Diagram showing inputs and outputs of the generalised body carbon model showing the isotopic composition (δ) and proportional fluxes (P) for inputs and outputs of the body carbon of an animal. Some fluxes that are considered negligible are not shown (e.g. dissolved carbon in drinking water, carbon inhaled as gas, carbon lost in urine).

FIG. 4 shows a comparison of carbon isotope ratios of wool from sheep that had their methane production abated via a supplement and those that did not. The difference is calculated relative to the average of the control group. Number of individual wool sample measurements: n=22 for each group.

FIG. 5 illustrates the more detailed rumen carbon model, where the carbon isotope ratios of inputs and outputs are indicated by δ, and the proportional flux of carbon indicated by P, with the subscript indicating the type of input or output.

FIG. 6 illustrates microbial processes in the rumen.

FIG. 7 illustrates the modelled change in carbon isotope ratios of ingested carbon (and animal tissues) of a methane abated animal relative to a control animal as the proportion of methane (P_{CH4}) is reduced as a percentage of the control. All other model parameters as in Table 1.

FIG. 8 illustrates a computer system for detecting methane reduction using methane reduction means on ruminant livestock.

### DESCRIPTION OF EMBODIMENTS

One way of demonstrating and quantifying a reduction of methane produced by livestock is measuring the concentrations of methane gas in breath. Some technology analyses exhaled methane concentrations in breath at a feeder and sends the data to the cloud, thus requiring internet connectivity. Other technology places each animal in individual respiration chambers for a period of measurement (e.g. 48 hours) and measures the methane emitted during that period. These methods are expensive, difficult to scale, and impractical for remote areas where internet is not readily available, and respiration chambers are unavailable. In addition, such methods are only usable on the animal for short periods rather than for the entire life-time of animal or duration while the animal was growing the product (as in wool). Further, these methods are not easily scaled to the entire livestock industry.

Reduced methane production in livestock due to the use of seaweed supplements (or other means) will shift the isotopic composition of animal breath and animal tissues. Using mass balance calculations and pilot data (see below), it was demonstrated that the reduction of methane production through seaweed supplements (or other means) will cause a shift in carbon isotope ratios on the order of 1-5% % or greater, which is detectable using conventional measurement techniques.

Therefore, carbon isotope ratios of the animal and of animal products (e.g. wool, meat, milk, leather, hair, hooves, bones, teeth, urine, faeces and all materials created by the animal) can be measured as a means of
Verifying reduced methane emissions in livestock due to application of seaweed supplements or other interventions;
Quantifying the proportional methane reduction due to application of seaweed supplements or other interventions;
Establishing the dosage of seaweed supplement or other interventions needed to achieve optimal methane reductions without over application;
Distinguishing phenotypic and genetic differences in methane production among breeds to enable selective breeding of low-emission breeds;
Certification of production systems (e.g. farms or collectives) with low-methane production through monitoring of animal tissues or animal waste;
Quantitative carbon accounting for industries that rely on methane producing livestock.

As an advantage, the disclosed method does not require on-farm technology, internet connectivity or large and expensive infrastructure. Further, the carbon isotopic shift associated with reduced methane emissions is inherent to the animal and all resulting product and outputs. Therefore, measurements can be conducted on the living animal (e.g. hair, breath), on animal waste (e.g. urine, faeces), on animal products (e.g. wool, meat, leather) or waste products (e.g. offcuts, waste wool). The measurement can also be conducted anywhere along the supply chain and does not need to be conducted on farm. Additionally, measurements can be conducted on different tissues within an animal with different turnover times (e.g. blood, hair, bone, muscle) to examine methane reduction on different timescales. The isotopic mark of a low-methane emission production methods persist throughout the supply chain from the farm to the point of sale and beyond.

### Method

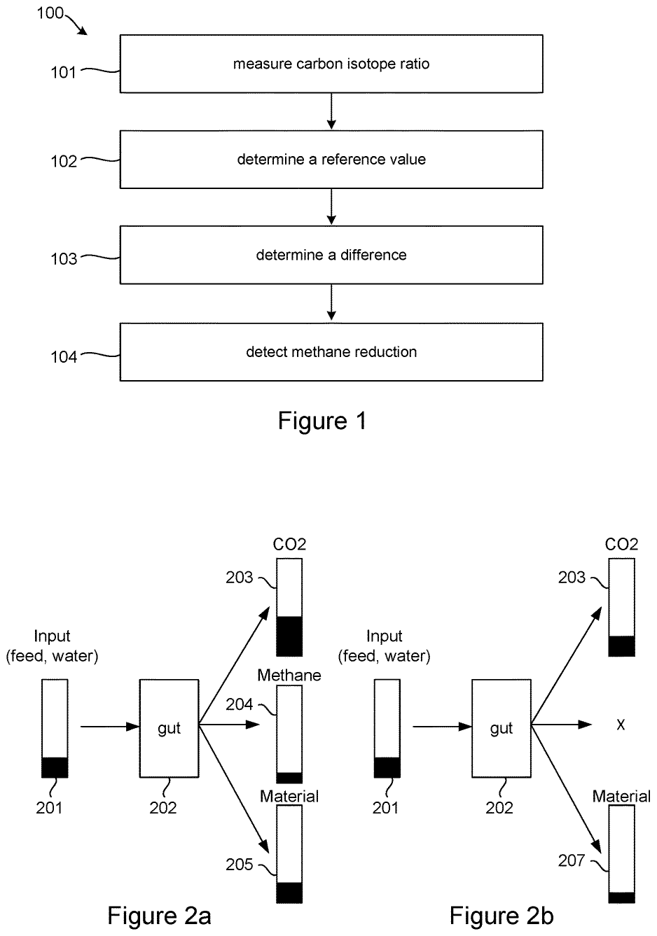

FIG. 1 illustrates a method 100 for detecting methane reduction using methane reduction means on ruminant livestock. The methane reduction means may comprise feeding a feed supplement, such as a seaweed supplement and in particular, Asparagopsis taxiformis. However, other reduction means can equally be applied, such as other supplements or other treatments or intervention strategies such as probiotics or forage quality types.

Method 100 comprises measuring 101 a carbon isotope ratio in material from the ruminant livestock under use of methane reduction means. That is, the ruminant livestock is subjected to the methane reduction means, such as being fed the feed supplement, and a product from the animal is sampled. This product can be a commercial product, such as wool, milk or meat, but may also be a non-commercial product, such as faeces, hooves, urine and others. The carbon isotope ratio is a ratio between the relative abundance of the stable isotopes 12C and 13C in a material relative to a standard and is reported in per mil units (% 0) as below in which R refers to the relative abundance of ¹³C/¹²C for the sample (sam) and the standard (std):

$${δ}^{13} C = ( [ ( {R}_{sam} ) / {R}_{std} ] - 1 ) \times 1000.$$

The carbon isotope ratios of materials vary in nature due to a range of chemical, biological and physical processes. It has been observed that when feed is consumed by a ruminant, the carbon isotope ratios of exhaled of CO₂ and belched CH₄ (herein considered both part of breath carbon) from a ruminant differ. More particularly, the CO₂ in the breath of the animal has a higher δ¹³C than the animal feed while the emitted methane has a lower ratio. Regardless, as set out in this disclosure, reducing the emissions in methane leads to a change in the ratio of ¹³C in the material from the animal. Therefore, in method 100 a measured value of the carbon isotope ratio is obtained from the material.

Then, method 100 determines 102 a reference value indicative of a reference carbon isotope ratio of material from the ruminant livestock without the use of the methane reduction means. This reference value is important because it provides a baseline from which to identify a change. The baseline can come from a variety of different sources. Firstly, the reference can be obtained from an animal. For example, the baseline can be obtained from a different animal of the same species that has been fed a comparable diet (without methane reducing supplement), or from an animal fed a quantifiably different diet (without methane reducing supplement) from which the δ¹³C offset of the reference can be adjusted to account for the difference in isotopic composition of the diet. That baseline measurement may be performed in the past (i.e. historical measurement) or at/around the same time that the material of the animal under methane abatement is measured.

Further, the reference value may be obtained from the same animal as the measured ratio but earlier and before the methane reductions means were used for that animal. Method 100 then determines 103 a difference between (a) the reference value, and (b) the measured value. Based on the difference, the method 100 detects 104 the methane reduction.

FIGS. 2a and 2b illustrate an animal uptake model without use of methane reduction means and with use of methane reduction means, respectively. It is noted that this uptake model is heavily simplified for illustrative purposes. A more detailed example is provided further below. The uptake model is essentially a linear mixing model because the methane that is not emitted is mixed into the remaining parts of the animal. In FIG. 2a, the input 201 consists of a high amount of ¹²C indicated by the white box and a low amount ¹³C indicated by the black box. The size of the boxes are chosen for illustrative purposes and do not represent real ratios as the ¹³C amount would be too small to be visualised meaningfully.

The input 201 may comprise feed, water, supplements etc. and enter the gut 202, where gut microbes produce methane and CO₂ and each gas has a different isotopic offset from the body carbon. As a result, the ratio of ¹³C differs between CO₂ 203, methane 204 and animal material 205. The mixing model may include a further range of materials, such as teeth, hooves, wool, milk, etc. but for the purposes of this disclosure it is sufficient to only consider the material that is being measured. The isotope ratio in the material 205 is not relevant as such, but serves as a baseline to detect any differences. What is significant, however, is there is relatively more ¹³C in CO₂ (larger black box in 203) than in the input 201 and relatively less 13C in methane (smaller black box in 204) than the input 201 and than in CO₂ (203).

FIG. 2b now illustrates the same animal species and the same isotope ratio in the input but now the animal is subject to methane reduction means, such as, the animal is being fed Asparagopsis to reduce methane. As a result, methane in FIG. 2b has been completely cancelled, noting that in most real examples there may be a small amount of methane still being emitted. However, since the methane 204 originally had a relatively low ¹³C amount, this low ¹³C/high ¹²C carbon is now absorbed in the animal material or exhaled in the CO₂. As a result, the relative amount of ¹³C in the animal material and in the CO₂ is lower compared to the relative amount without methane reduction. This can be seen in FIG. 2b at 207 indicating the reduced relative amount of ¹³C visualised by the smaller black box compared to FIG. 2a. So the material ratio 205 in FIG. 2a serves as the reference value or baseline and the ratio 207 in FIG. 2b serves as the measurement from which the methane reduction can be detected.

As mentioned above, the reference can be obtained from a different individual animal as long as its diet is either the same or the differences can be quantified in terms of isotopic signature. More particularly, the diet of the reference animal may be similar in relation to its carbon isotope ratio, where “similar” means within the accuracy that is desired for the calculation output, such as 10% or 5%. In other examples, the diet is similar in abundance of C3 plant and C4 plant components. This is achievable since certain feeding areas promote the growth of predominantly one type of plants and therefore, as long as that reference animal is raised in the same region, the feed is likely to be similar (within 5% or 10%) in C3/C4 plants to the test animal. Therefore, such a reference is relatively easily obtainable. In addition, reporting of different proportions of animal feed and their isotopic ratios can be used within a mixing model to calculate the overall isotopic composition of the diet, to enable the correction of the reference for any isotopic differences in diet.

It is again noted that the reference value may be the reference carbon isotope ratio from a first tissue that is of a different tissue type than a second tissue from which the measured carbon isotope value is obtained. So the reference may be from hooves or hair grown before or after methane reduction, while the actual measurement is from meat or other material produced during methane reduction. Again, there may be an offset in the calculations to account for the different uptake of carbon isotopes in different materials which is constant over time and constant across different individuals of the animal species.

In a further example, the disclosed method estimates the diet using geographic parameters. That is, the method may have access to a geographical distribution of the carbon isotope ratio of the vegetation. The method then receives the location or area where the animal was fed on locally grown grass, grain or silage. Accordingly, the method can evaluate a plant update model to calculate an estimated carbon isotope ratio of the input to the animal mixing model. Alternatively, the method may receive the area where the animal feed was grown and estimates the carbon isotope ratio from that information.

In a further example, the difference between the reference ratio 205 and the measured ratio 207 can be used to adjust the amount of supplement fed to the ruminant livestock to achieve a desired detection or quantity of reduced methane. That is, the material can be measured multiple times during the life of the animal. For example, the carbon isotope ratio in milk can be tested relatively frequently. This provides an indication of a current methane reduction in the animal as a result of adding a feed supplement. As the amount of supplement is reduced, the methane reduction may also decrease. For certification purposes, the reduction may need to exceed a predefined value, such as 95%. Therefore, the supplement amount can be adjusted so that the animal remains above 95% reduction without wasting supplement for limited benefit.

As such, this disclosure further provides a method for feeding a supplement to ruminant livestock at a dosage that causes a change in carbon isotope ratio in material from the animal indicative of a reduction in methane. That is, the supplement is fed so that a methane reduction is detectable using the disclosed method. In some examples, the supplement may be fed at a relatively low rate that does not lead to detection of methane reduction. The amount of supplement is then increased and the material from the animal is repeatedly measured until a methane reduction is detectable. At this stage, the amount of supplement can be adjusted to achieve a desired reduction. The resulting amount of supplement for particular animal species and potentially particular main food source can be provided as instructions in a kit together with the actual supplement, which may contain Asparagopsis to be given at the determined amount.

The methods disclosed herein can further be used to establish a phenotype that can be used for selective breeding, directly or through breeding values. More specifically, the methods disclosed herein are used to determining for multiple individuals an amount of methane produced, which is the phenotype that is used in genetic and/or genomic evaluation. Then, according to the resulting breeding values, individual animals are selected for further breeding based on the amount of methane produced either through natural variations in animals who are not fed supplements, or those that are more susceptible to reductions in emissions in response to supplement. In the first case, selection of low-methane emitting animals could be used to develop lower emissions herds. In the second case, selection could be done to improve the overall herd efficiency of converting feed supplement to methane reduction and may reduce the amount of supplement required to achieve an almost 100% (below measurable) reduction in methane reduction.

Provided herein is also an animal that has been bred according to the method above. That is, the bred animal has particular characteristics in uptake of the feed supplement with a higher reduction in methane for the same amount of supplement compared to another population of animals.

Finally, there is provided an animal product having a measured relative carbon isotope ratio difference indicative of a reduced methane emission as a result of feeding a supplement to the animal. For example, this may be a commercial product, such as wool, meat, milk or other product, that has been measured and the measured isotope ratio indicates that methane emissions have been reduced. This measurement can be provided by an independent body and the product can be labelled accordingly, which may attract consumer confidence and willingness to purchase the product or goods made from such a product. Overall, this generates a market mechanism that provides for an incentive to use the methods disclosed herein. More particularly, there may be a certification body that requires a minimum amount of measured methane reduction with a reliable and repeatable process. Once all requirements are met, the certification body certifies the animal product to have reduced methane emissions. This certification can be later confirmed by performing the same measurement again, which provides a good opportunity for controlling the supply chain and preventing fraud through falsely labelled products.

### Mass Balance Calculations to Illustrate the Effect of Reduced Methane Emissions

As described above, using mass balance calculations for domestic ruminants (e.g., sheep and cattle) the effect of reducing methane emissions by seaweed (Asparagopsis) supplements or other means can be demonstrated by using a few assumptions and values from the literature for key parameters.

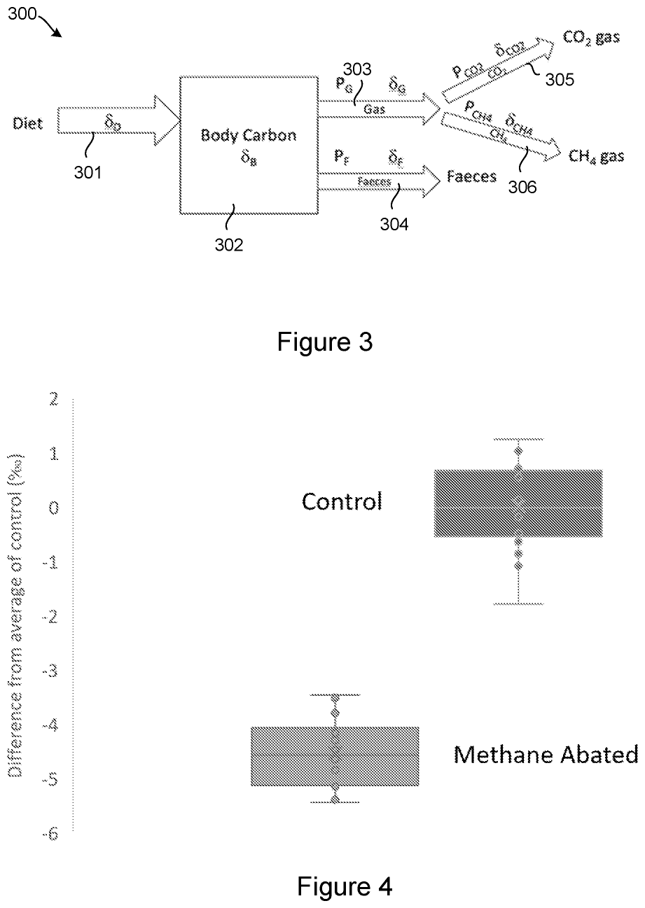

FIG. 3 illustrates another example for a body carbon model comprising an input 301 through diet, a body carbon element 302, gas output 303 and faeces output 304. The body carbon element 302 in this example includes all material that may be obtained from the animal for a commercial purpose, such as meat, milk, wool, etc. The carbon isotope ratios of inputs 301 and outputs 303/304 are indicated by δ with the subscript indicating the type of input or output, and the proportional flux of carbon indicated by P for each output. Note that the gas output 303 is divided into a CO₂ 305 and a CH₄ component 306, each with their own proportional flux and isotopic composition.

The isotopic compositions of the outputs can be represented as fractionations (offset) relative to the body carbon isotopic composition. This fractionation is represented by Δ_{a-b} in which the subscript denotes the two materials (a and b for example). In the discussion below the following subscripts are used for all terms: D for diet, B for body, G for gas, F for faeces, CO₂ for CO₂ and CH₄ for CH₄.

At steady state the following represents the relationships between inputs and outputs.

$${δ}_{D} = {P}_{G} \cdot ( {δ}_{B} + {Δ}_{G - B} ) + {P}_{F} \cdot ( {δ}_{B} + {Δ}_{F - B} ) \qquad ( Equation 1 )$$

$${P}_{F} = 1 - {P}_{G} \qquad ( Equation 2 )$$

Therefore, equation 1 can be rearranged as follows.

$${δ}_{D} = {δ}_{B} + {P}_{G} \cdot {Δ}_{G - B} + {P}_{F} \cdot {Δ}_{F - B} \qquad ( Equation 3 )$$

The offset between the gas and body isotopic composition (Δ_{G-B}) reflects the relative abundance of the flux as CO₂ and as CH₄.

$${Δ}_{G - B} = {P}_{CO 2} ( {Δ}_{CO 2 - B} ) + {P}_{CH 4} ( {Δ}_{CH 4 - B} ) \qquad ( Equation 4 )$$

The offset between CO₂ and CH₄ from the same animal is measured to be −50% 0. This can be approximately represented as in equation 5.

$${Δ}_{CH 4 - B} = {Δ}_{CO 2 - B} - 50 {\%}_{0} \qquad ( Equation 5 )$$

By substituting equation 5 and equation 6 into equation 4, it can be rearranged to yield equation 7.

$${P}_{CH 4} = 1 - {P}_{CO 2} \qquad ( Equation 6 )$$

$${Δ}_{G - B} = {Δ}_{CO 2 - B} + {P}_{CH 4} \cdot - 50 \text{?} \qquad ( Equation 7 )$$

Therefore, by substitution equation 7 into equation 3 and rearranging, the following equation is reached.

$${δ}_{B} = {δ}_{D} - {P}_{G} [ {Δ}_{CO 2 - B} + {P}_{CH 4} \cdot - 50 \text{?} ] - {P}_{F} \cdot {Δ}_{F - B} \qquad ( Equation 8 )$$

For most cases of comparing within a species several terms in equation 8 will remain constant. These are the proportion of carbon lost as gas and faeces (P_{G} and P_{F}) and the offsets between the body and faeces (Δ_{F-B}) and body and CO₂ (Δ_{CO2-B}). In the subsequent calculations, these are assumed to be constant, however, variations of these factors can also be accounted for within this model. For cases in which the terms listed above can be considered constant, equation 8 demonstrates that the body isotopic composition (δ_{B}) will depend only on the isotopic composition of the diet (δ_{D}) and the proportion of carbon lost as CH₄ (P_{CH4}).

Two materials of the same type (e.g. wool, muscle, bones, blood, milk, faeces) with a constant fractionation relative to δ_{B} can be compared to establish a change in δ_{B}. This change in δ_{B} between a sample(S) and a reference (R) as calculated by the difference of two values of the same animal material is here termed ΔB_{S-R}.

$$Δ {B}_{S - R} = {δ}_{B ( S )} - {δ}_{B ( R )} \qquad ( Equation 9 )$$

The difference between the diet of the sample and reference animal is calculated as ΔD_{(S-R)}.

$$Δ {D}_{S - R} = {δ}_{D ( S )} - {δ}_{D ( R )} \qquad ( Equation 10 )$$

By recognising that P_{G}, P_{F}, Δ_{F-B} and Δ_{CO2-B} will likely remain constant for both the reference and sample animal, the following equation is reached that illustrates the importance of P_{CH4} for the sample and reference (P_{CH4(S)} and P_{CH4(R)}, respectively).

$$Δ {B}_{S - R} - Δ {D}_{S - R} = {P}_{G} [ - 50 \text{?} \cdot ( {P}_{CH 4 ( R )} - {P}_{CH 4 ( S )} ) ] \qquad ( Equation 11 )$$

The term ΔB_{S-R}-ΔD_{S-R} represents the isotopic difference between the sample and reference materials that is not due to differences in isotopic composition of the diet. Equation 11 demonstrates that the diet-adjusted difference (ΔB_{S-R}-ΔD_{S-R}) is directly proportional to the difference between the reference and sample in their methane emissions proportion P_{CH4}.

To illustrate the change in methane emissions proportion (x-axis, FIG. 4) the differences in reference and sample diets (ΔD_{S-R}) and animal material (ΔB_{S-R}) is shown.

In the case of feed supplements, the isotopic composition of the supplement may contribute to the ΔD_{S-R} value and can serve as an additional marker of supplements being used. This dietary effect would be calculated using a mixing model and the proportion of supplement in the feed and the isotopic composition of that supplement relative to the rest of the diet.

Therefore, the difference between the isotopic composition of body carbon for a ruminant who has had methane reduction can be identified through the use of the carbon isotope composition of animal materials in conjunction with information about the isotopic composition of its diet.

Further detail on carbon isotope ratios of enteric methane is provided in: Klevenhusen, F., Bernasconi, S. M., Kreuzer, M., & Soliva, C. R. (2010). Experimental validation of the Intergovernmental Panel on Climate Change default values for ruminant-derived methane and its carbon-isotope signature. Animal Production Science, 50(3), 159-167. doi: https://doi.org/10.1071/AN09112, which is incorporated herein in full by reference.

### Detailed Rumen Carbon Model

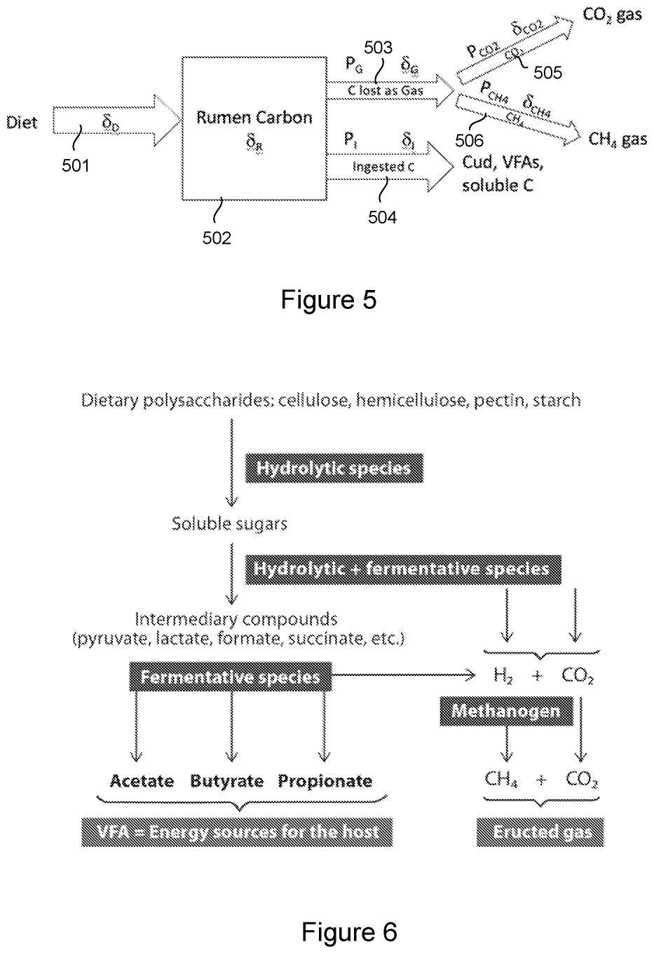

A more detailed and specific model can be developed for the rumen, where the microbial production of methane occurs. In the rumen carbon model shown in FIG. 5, the carbon isotope ratios of inputs and outputs are indicated by δ, and the proportional flux of carbon indicated by P for each output, with the subscript indicating the type of input or output. It is noted that the methods disclosed herein may use the model in FIG. 3 or the model in FIG. 5 interchangeably, noting that the model in FIG. 3 provides an overview of the body as a whole, while that in FIG. 5 relates specifically to the isotopic effects within the rumen and the carbon ingested from the rumen.

In FIG. 5, the influx of carbon is via the diet 501 to the rumen 502 and the outflux of carbon is split between the proportion that the proportion that is lost as gas (P_{G}; 506) and the proportion that is ingested (P_{I}; 504). The loss as gas is further divided into the proportion lost as carbon dioxide (P_{CO2}; 505) or the proportion lost as methane (P_{CH4}; 506). Note that the CO₂ lost from the rumen is from microbial fermentation not animal respiration.

Carbon isotope ratios are expressed in delta notation as

$${δ}^{13} C = {R}_{sample} / {R}_{standard} - 1 \qquad ( Equation 12 )$$

$$R = [ {}^{13} C ] / [ {}^{12} C ] \qquad ( Equation 13 )$$

R represents the relative abundance of ¹³C to ¹²C in the sample and a standard. The standard for reporting carbon isotope ratios is Vienna Pee Dee Belemnite (VPDB). Carbon isotope ratios are routinely reported in permil (% 0), or parts per thousand.

At steady state, the carbon isotope ratio of the dietary influx (δ_{D}) is equal to the proportionally weighted carbon isotope ratio of the outflux as ingested carbon (δ₁) carbon lost as carbon dioxide (δ_{CO2}) and methane (δ_{CH4}). This weighting can be represented in a mass balance equation.

$${δ}_{D} = {P}_{1} \times {δ}_{1} + {P}_{G} \times ( {P}_{CO 2} \times {δ}_{CO 2} + {P}_{CH 4} \times {δ}_{CH 4} ) \qquad ( Equation 14 )$$

This equation can also be expressed in terms of ¹³C/¹²C abundance ratios (R) as follows.

$${R}_{D} = {P}_{1} \times {R}_{1} + {P}_{G} \times ( {P}_{CO 2} \times {R}_{CO 2} + {P}_{CH 4} \times {R}_{CH 4} ) \qquad ( Equation 15 )$$

Given that P_{G} and P_{I} sum to one, and P_{CO2} and P_{CH4} sum to one, substitution lead to equation 17.

$${P}_{G} = 1 - {P}_{I} \qquad ( Equation 16 )$$

$${P}_{CO 2} = 1 - {P}_{CH 4} \qquad ( Equation 17 )$$

$${R}_{D} = {P}_{I} \times {R}_{I} + ( 1 - {P}_{I} ) \times ( ( 1 - {P}_{CH 4} ) \times {R}_{CO 2} + {P}_{CH 4} \times {R}_{CH 4} ) \qquad ( Equation 18 )$$

Microbial processes in the rumen produce CO₂ and then transform that CO₂ into CH₄ (FIG. 6). The isotopic fractionation during the production of CH₄ is expressed as a fractionation factor, alpha (a) and is considered constant.

$${α}_{CH 4 - CO 2} = {R}_{CH 4} / {R}_{CO 2} \qquad ( Equation 19 )$$

Similarly, the relationship between the isotope ratio of the ingested carbon and the microbial CO₂ can be expressed as a fractionation factor (α) and is considered constant.

$${α}_{I - CO 2} = {R}_{I} / {R}_{CO 2} \qquad ( Equation 20 )$$

Therefore, the relationship between the isotopic ratio of carbon entering the rumen via diet (R_{D}) and that exiting as CO₂ can be expressed as a function of the proportion of methane produced as follows.

$${R}_{D} = {P}_{I} \times {R}_{CO 2} \times {α}_{I - CO 2} + ( 1 - {P}_{I} ) \times ( ( 1 - {P}_{CH 4} ) \times {R}_{CO 2} + {P}_{CH 4} \times {R}_{CO 2} \times {α}_{CH 4 - CO 2} ) \qquad ( Equation 21 )$$

$${R}_{CO 2} = {R}_{D} / [ {P}_{I} \times {α}_{I - CO 2} + ( 1 - {P}_{I} ) \times ( 1 - {P}_{CH 4} + {P}_{CH 4} \times {α}_{CH 4 - CO 2} ) ] \qquad ( Equation 22 )$$

The isotope ratio of the ingested carbon can then be calculated from the isotope ratio of rumen CO₂ estimated in equation 22 by rearranging Equation 20 to produce equation 23.

$${R}_{I} = {α}_{I - CO 2} \times {R}_{CO 2} \qquad ( Equation 23 )$$

Changes to the carbon isotope ratio of ingested carbon due to changes in methane production will be passed on to the tissues that are formed in the animal. Therefore, this mass balance model enables the isotopic composition animal tissues to be related to changes in their methane production. To quantify this relationship the following terms need to be estimated or measured: P_{G} and P_{I}; P_{CH4} and P_{CO2}; and α_{CH4-CO2}. The following worked example demonstrates how these values can be estimated from the existing scientific literature.

Results from in vitro fermentation experiments can be used estimating terms in the rumen model because they eliminate the contribution from animal respired CO₂. From such studies we can estimate of several relevant fluxes (P) and fractionation factors (α).

Estimation of the proportion of C lost from the rumen as gas (P_{G}) from in vitro fermentation as the sum of CH₄ and CO₂ in ml/gDM (dry matter), is estimated as 250 ml/gDM, or 0.011 moles C/gDM, or 0.132 g C/gDM. The dry matter ration is here assumed to be 40% C enabling estimation of the proportion of carbon lost as gas as 0.132 g C/0.4 g C, or 0.33. The example below therefore presumes a split of 33% gas (P_{G}) and 67% ingested (P_{I}).

In vitro fermentation gas production measurements, stoichiometric considerations and modelling of fermentation by rumen microbiota suggest carbon lost from the rumen as gas is divided roughly as 30% CH₄ (P_{CH4}), and 70% CO₂ (P_{CO2}).

The fractionation factor between CH₄ and CO₂ (α_{CH4-CO2}) can be estimated from isotope ratios of gases from in vitro incubations of rumen fluids and expressed also as an epsilon value expressed in permil (Table 1).

$$ε = α - 1 \qquad ( Equation 24 )$$

The fractionation factor between ingested carbon and CO₂ (α_{I-CO2}) can be estimated by the rumen carbon model using the values estimated here for P_{I}, P_{G}, P_{CO2} and P_{CH4} from in vitro fermentation experiments and measured isotope ratios of diet, CO₂ and CH₄ from animals that have not had any methane abatement (Table 1).

TABLE 1
Parameter values used to demonstrate model

| Fractionations | ε (‰) | α |
|---|---|---|
| CO₂-diet | 19.0 | 1.0190 |
| CH₄—CO₂ | −62.1 | 0.9379 |
| I-CO₂ | −18.7 | 0.9813 |
| Control conditions: | Delta (δ) | Rₛₐₘ/R_{std} |
| δ_{D} | −28 | 0.972 |
| δ_{CO2} | −9.5 | 0.990468 |
| δ_{CH4} | −71.0 | 0.92896 |
| δ_{I} | −28.0 | 0.97199 |
| P_{I} | 0.67 | |
| P_{G} | 0.33 | |
| P_{CO2} | 0.7 | |
| P_{CH4} | 0.3 | |

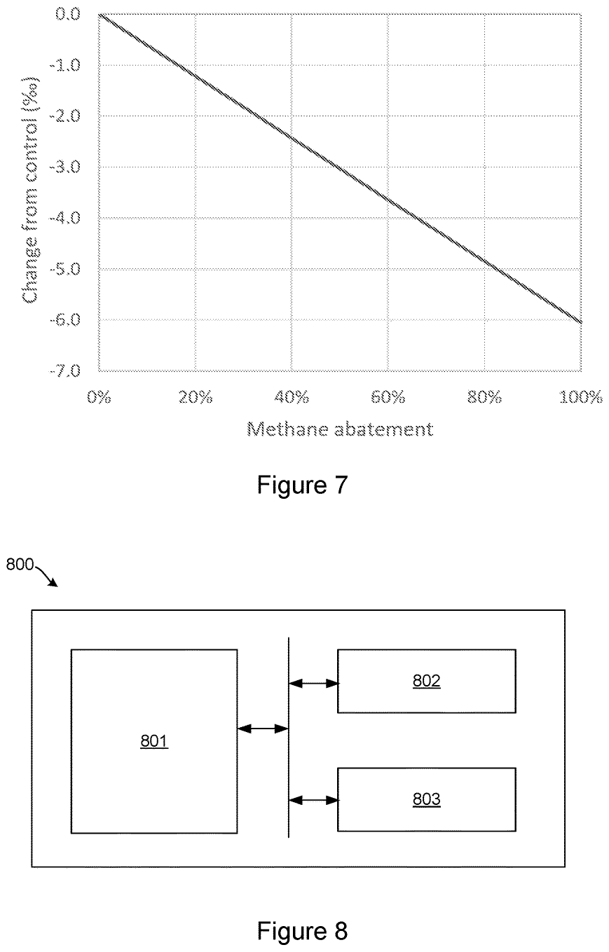

Using these values (Table 1), the effect of changes in the proportion of carbon lost as methane on the carbon isotope ratio of ingested carbon can be modelled. In the worked example here, only P_{CH4} and P_{CO2} are allowed to vary while all other parameters are held constant at the values in Table 1. The change in carbon isotope ratios of ingested carbon in an abated animal relative to a control (unabated) animal is directly proportional to the degree of methane abatement (FIG. 7). Any changes in the isotope ratio of ingested carbon will also be recorded in the animal tissues synthesized using that ingested carbon.

In FIG. 7, the isotope ratio of the diet is held constant between the control and methane abated animals. However, control and methane abated animals with different diets can also be used for comparison by accounting for difference in the carbon isotope ratio of the diets. Similarly, other parameters could be changed or allowed to vary in the application of the model. The model describes the relationship between carbon isotope ratios, fluxes and methane production in a way that enables quantification of differences in methane production among and within animals.

### Isotopic Verification and Quantification of Methane Abatement in Livestock

The implication of the estimates in the above calculations and FIG. 4 is that methane abatement of the type achieved by seaweed supplements or other means should cause a shift in the carbon isotope ratio of animal tissues, breath and excretions on the order of −1 to −5% %, which is more than ten times the measurement precision routinely achieved in commercial labs. This effect imparts a clear signature of methane abatement in the animal tissues and products (e.g. wool, meat, leather, hair, milk, blood, bone, faeces etc.).

To demonstrate proof of concept, carbon isotope ratios of wool was measured from sheep that had been fed a methane reducing supplement (Asparagopsis) to abate their methane production, and those that were not, but were otherwise fed the same diet. The results (FIG. 4) show a clear distinction in the carbon isotope ratio of the wool. The average difference is −4.5±1% %, which matches predictions for sheep of roughly 70% abatement in the worked example of the model (FIG. 7).

### Computer System

FIG. 8 illustrates a computer system 800 for detecting methane reduction by using methane reduction means on ruminant livestock. Computer system 800 comprises a processor 801 to perform the methods disclosed herein, including the method 100 in FIG. 1. Processor 801 may be implemented as a single processor or multiple processors. Processor 801 may also be implemented as part of a distributed (cloud) computing environment. Computer system 800 may be a server, on-field or on-farm edge computer, personal computer, mobile communication device or other computer system. Computer system 800 further comprises non-volatile, i.e. non-transitory computer readable medium 802, with program code stored thereon. The program code, i.e. software, causes processor 801 to perform the methods disclosed herein.

Computer system 800 further comprises data memory 803 to store data values of the model, measurement values, output values or other values. Data memory 803 may also be implemented as a database, external database, cloud storage and other data memory types.

By way of software stored on program memory 802, processor 801 receives a measured value indicative of a measurement of a carbon isotope ratio in material from the ruminant livestock under use of the methane reduction means. Processor 801 then determines a reference value indicative of a reference carbon isotope ratio of material from the ruminant livestock without the use of the methane reduction means. Then, the processor 801 determines a difference between (a) the reference value, and (b) the measured value; and based on the difference, detects the methane reduction. It is noted that the optional features described herein can also be performed by processor 801. The computer system 801 may further be coupled to a display device that displays an indication of methane reduction to a user.

### Practical Applications

Practical application of carbon isotope differences for verifying and quantifying methane abatement is based on a baseline value of a non-abated animal of the same diet for comparison. This value can be obtained empirically (measurement of baseline un-abated animals prior to, or co-eval with using seaweed supplements for abatement) or theoretically (modelling C3 and C4 in diet, reporting on diet, and modelling the non-abated value adjusted for any differences in diet). Existing models of rangeland vegetation can be used to predict forage composition by location. Example models include:
Munroe, S. E. M., Guerin, G. R., McInerney, F. A., Martín-Forés, I., Welti, N., Farrell, M., . . . Sparrow, B. (2022). A vegetation carbon isoscape for Australia built by combining continental-scale field surveys with remote sensing. Landscape Ecology. doi:10.1007/s10980-022-01476-y
Munroe, S. E. M., McInerney, F. A., Andrae, J., Welti, N., Guerin, G. R., Leitch, E., . . . Sparrow, B. (2021). The photosynthetic pathways of plant species surveyed in Australia's national terrestrial monitoring network. Scientific Data, 8(1), 97. doi:10.1038/s41597-021-00877-z

Both of which are included herein by reference.

Additionally, different animal tissues compared to one another formed at different times could be used to examine changes in diet throughout the life of the animal, prior to and after seaweed supplementation, for example.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A method for detecting methane reduction by using methane reduction means on ruminant livestock, the method comprising:
measuring a carbon isotope ratio in material from the ruminant livestock under use of the methane reduction means, to obtain a measured value;
determining a reference value indicative of a reference carbon isotope ratio of material from the ruminant livestock without the use of the methane reduction means;
determining a difference between
(a) the reference value, and
(b) the measured value; and
based on the difference, detecting the methane reduction.

2. The method of claim 1, wherein determining the reference value comprises measuring the reference carbon isotope ratio from the same individual from which the measured value is obtained on material grown at a time without the use of the methane reduction means.

3. The method of claim 1, wherein determining the reference value comprises measuring the reference carbon isotope ratio from a different individual that has been fed a comparable diet to the ruminant livestock without the use of the methane reduction means, wherein the comparable diet is similar in relation to its carbon isotope ratio and/or abundance of C3 plant and C4 plant components.

4. (canceled)

5. The method of claim 1, wherein determining the reference value comprises:
measuring the reference carbon isotope ratio from an animal with a different diet; and
correcting the reference carbon isotope ratio for the different diet by calculating an isotopic composition of the diet and adjusting the reference carbon isotope ratio based on the isotopic composition.

6. The method of claim 1, wherein determining the reference value comprises measuring the reference carbon isotope ratio from a first tissue that is of a different tissue type than a second tissue from which the measured carbon isotope value is obtained, wherein the method further comprises applying an offset to account for a constant difference in carbon isotope uptake in different tissues.

7. (canceled)

8. The method of claim 1, wherein determining the reference value comprises evaluating an uptake model, and an input to the uptake model comprises a diet of the ruminant livestock without the use of the methane reduction means, wherein the method further comprises estimating a diet using geographic parameters.

9. (canceled)

10. The method of claim 1, wherein the material comprises one or more of: meat, wool, excrement, milk, bones, leather, hair, reproductive material or skin.

11. The method of claim 1, wherein the method further comprises evaluating a system model of the ruminant livestock.

12. The method of claim 11, wherein the system model is indicative of relative abundance of carbon isotopes entering the ruminant livestock and relative abundance of carbon isotopes exiting the ruminant livestock during the life of the ruminant livestock and relative abundance of carbon isotopes remaining in the material of the ruminant livestock.

13. The method of claim 11, wherein the system model is indicative of relative abundance of carbon isotopes entering the ruminant livestock and relative abundance of carbon isotopes exiting the ruminant livestock as gas during the life of the ruminant livestock and relative abundance of carbon isotopes ingested by the ruminant livestock.

14. The method of claim 1, wherein the method further comprises calculating a quantity of methane reduction based on the difference, wherein calculating the quantity comprises evaluating a linear relationship representing a mixing model of carbon isotopes in a body of the ruminant livestock.

15.-17. (canceled)

18. A method for feeding a supplement to ruminant livestock at a dosage that causes a change in carbon isotope ratio in material from an animal indicative of a reduction in methane.

19. The method of claim 18, wherein the supplement comprises Asparagopsis.

20. A method to establish a phenotype for genetic and/or genomic selection, the method comprising:
determining for multiple individuals the phenotype for the amount of methane produced according to any one of the preceding claims;
use the phenotype in genetic and/or genomic evaluation; and
selecting individuals for further breeding based on the breeding values for methane emission.

21. An animal bred according to the method of claim 20.

22. A kit comprising a feed supplement and instructions to feed the feed supplement to ruminant livestock for detection of methane emission according to claim 1.

23. An animal product having a measured relative carbon isotope ratio difference indicative of a reduced methane emission as a result of feeding a supplement to the animal.

24. An animal product certified according to the method of claim 1.

25. A computer system for detecting methane reduction by using methane reduction means on ruminant livestock, the computer system comprising one or more processors configured to:
receive a measured value indicative of a measurement of a carbon isotope ratio in material from the ruminant livestock under use of the methane reduction means;
determine a reference value indicative of a reference carbon isotope ratio of material from the ruminant livestock without the use of the methane reduction means;
determine a difference between
(a) the reference value, and
(b) the measured value; and
based on the difference, detect the methane reduction.

26. A non-transitory computer readable medium with program code stored thereon that, when executed by a computer, causes the computer to perform a method for detecting methane reduction by using methane reduction means on ruminant livestock, the method comprising:
receiving a measured value indicative of a measurement of a carbon isotope ratio in material from the ruminant livestock under use of the methane reduction means;
determining a reference value indicative of a reference carbon isotope ratio of material from the ruminant livestock without the use of the methane reduction means;
determining a difference between
(a) the reference value, and
(b) the measured value; and
based on the difference, detecting the methane reduction.
